(19) United States
(12) Patent Application Publication
(10) Pub. No.: US 2005/0227998 A1
(43) Pub. Date: Oct. 13, 2005

(54) Compositions and methods of decreasing nicotine withdrawal

(76) Inventor: Kirk Voelker, Sarasota, FL (US)

Correspondence Address: MCDERMOTT WILL & EMERY LLP, 600 13th Street, N.W., Washington, DC, 20005-3096, US

(21) Appl. No.: 10/883,972

(22) Filed: Jul. 6, 2004

Related U.S. Application Data
(63) Continuation of application No. 10/709,091, filed on Apr. 13, 2004.

(51) Int. Cl. A61K 31/495; A61K 31/46

(52) U.S. Cl. 514/255.04; 514/304

(57) ABSTRACT

Administering to a patient an anticholinergic medication comprising scopolamine and hydroxyzine can alleviate the symptoms of nicotine withdrawal. Used in conjunction with pre- and post-administration counseling, smoking cessation rates may be significantly improved.

This application is a continuation and claims the benefit of U.S. patent application Ser. No. 10/709,091, filed on Apr. 13, 2004, which is herein incorporated by reference.

### FIELD OF INVENTION

This invention relates to compositions and methods for the treatment of nicotine withdrawal symptoms associated with smoking cessation.

### BACKGROUND OF THE INVENTION

Cigarette smoking is the number one cause of preventable morbidity and mortality in the United States. Nicotine addiction is one of the most difficult addictions to overcome with recidivism equaling that of heroin addiction. The duality of nicotine addiction leads to both a physiologic addiction and a psychological addiction.

The physiologic principals of nicotine indicate that nicotine acts as an agonist at nicotinic receptors in the parasympathetic nervous system. The highest concentration of nicotine receptors resides centrally in the mid-brain. With chronic nicotine use there is a chronic block of nicotine receptors. In response to this chronic blockade of the nicotine receptors, there is a compensatory increase of acetylcholine through a process of enzyme induction of acetylcholine transferase. Therefore, when a smoker quits smoking, there is a withdrawal of this nicotine blockade and a marked increase of the neurotransmitter acetylcholine. This unchecked abundance of acetylcholine is in part responsible for the physiologic withdrawal of smoking cessation. Anticholinergic agents block the muscarinic receptors and attenuate the symptoms of nicotine withdrawal.

Glick, et al. have found that scopolamine decreased smoking patterns in monkeys. Glick S D, Jarvick M E, Nakamura NK Inhibition by Drugs of Smoking Behavior in Monkeys Nature 227:969:71 (1970) Bachynsky has demonstrated a significant improvement in smoking cessation rates of 500 smokers by using a solution of atropine and scopolamine potentiated by chlorpromazine injected subcutaneously and behind each ear. Brachynsky N. The Use of Anticholinergic Drugs fro Smoking Cessation: a Pilot Study Int J Addiction 21(7): 789-805 (1986) U.S. Pat. No. 4,555,397. Atropine, an anticholinergic, has an adverse side effects of causing an increased heart rate and thus, heightened risk of cardiac arrhythmias. Chlorpromazine, while not anticholinergic, does have anxiolytic properties. However, chlorpromazine also has a suboptimal side effect profile including dystonic extrapyramidal effects, tardive dyskinesia, hypotension, neuroleptic malignant syndrome, seizures, QT prolongation and torsades de pointes and is relatively contraindicated in patients taking popular serotonin uptake inhibitors for depression. As a result, there is a need for more safe efficacious treatments for nicotine withdrawal.

We have found that an anticholinergic injection of scopolamine and hydroxyzine is equally effective to the triple combination of atropine, scopolamine and chlorpromazine with a better side effect profile.

### SUMMARY OF THE INVENTION

The present invention provides compositions and methods that are useful for treating nicotine withdrawal symptoms associated with smoking cessation. The present invention is based upon the discovery that the combination of scopolamine and hydroxyzine can be effectively administered together or substantially together for enhanced therapeutic treatment of nicotine withdrawal symptoms associated with smoking cessation.

In one aspect of the invention there are anticholinergic medications for the treatment of nicotine withdrawal comprising a therapeutically effective amount of scopolamine and hydroxyzine. Optionally, the anticholinergic medications further comprise a therapeutically effective amount of one or more of an anticholinergic agent, preferably selected from the group consisting of atropine, belladonna, chlorpromazine, homatropine, hyoscyamine and glycopyrrolate. In another preferred embodiment, the anticholinergic medication further comprises a therapeutically effective amount of hyoscyamine. In yet another preferred embodiment, the anticholinergic medication further comprises a therapeutically effective amount of glycopyrrolate. The anticholinergic medication may be administered simultaneously or substantially together. Preferably, the anticholinergic medication is administered by injection in a pharmaceutically acceptable carrier.

In another aspect of the invention, the present invention comprises a method of minimizing nicotine withdrawal symptoms in a patient in need thereof comprising, administering to the patient a therapeutically effective amount of an anticholinergic medication comprising scopolamine and hydroxyzine. Optionally, the method comprises the anticholinergic medication further comprising an effective amount of one or more of an anticholinergic agent, preferably selected from the group consisting of atropine, chlorpromazine, homatropine, hyoscyamine and glycopyrrolate.

In one embodiment of the invention, the method of minimizing nicotine withdrawal symptoms further comprises the step of prescribing, concomitantly, one or more of a therapeutic dose of an anticholinergic agent using accepted pharmacological principals for use by the patient. In a preferred embodiment the anticholinergic agent is selected from the group consisting of atropine, belladonna, donnatal, homatropine, hydroxyzine, hyoscyamine, glycopyrrolate, oxybutynin, propantheline and scopolamine.

In yet another embodiment of the invention, the method of minimizing nicotine withdrawal symptoms further comprises a step of reducing anxiety by prescribing, concomitantly, one or more of a therapeutic does of an anxiolytic agent preferably comprising, benzodiazepines, chlorpromazine, clonidine and/or hydroxyzine for use by the patient.

In still another embodiment, the method of the present invention further comprises prescribing a therapeutic dose of an antidepressant. Preferably, the antidepressant is a serotonin reuptake inhibitor, bupropion or buspirone. The antidepressant may be prescribed and utilized by the patient to prior to, in conjunction with, or following administration of the anticholinergic medication.

In still another embodiment, the method of minimizing nicotine withdrawal symptoms further comprises a step of implementing behavioral modification therapy.

### BRIEF DESCRIPTION OF THE DRAWINGS

FIG. 1(A) is a table showing the success rate of 3 groups of patients one month after having received the claimed invention.

FIG. 1(B) is a bar graph representation of the data in FIG. 1 (A).

### DETAILED DESCRIPTION

All patents, patent applications and literature cited in this description are incorporated herein by reference in their entirety. In the case of inconsistencies, the present disclosure, including definitions, will prevail.

This invention provides a therapeutically effective anticholinergic medication useful for the treatment of nicotine withdrawal symptoms. In a preferred embodiment, the anticholinergic medication comprises therapeutically effective amounts of scopolamine and hydroxyzine. Scopolamine is an alkaloid drug obtained from plants of the nightshade family (Solanaceae), but primarily from henbane, Hyoscyamus niger. Structurally similar to the nerve substance acetylcholine, scopolamine acts by interfering with the transmission of the nerve substance acetylcholine in the parasympathetic nervous system. The compositions of the present invention for the treatment of nicotine withdrawal symptoms comprise formulations about 0.25 cc to about 6 cc in volume, wherein scopolamine is present in an amount ranging from about 0.01 mg to about 0.8 mg.

Hydroxyzine is a piperazine derivative antihistamine which has anticholinergic, anxiolytic, antiemetic, antispasmodic and local anesthetic activity. Suitable compounds of hydroxyzine include by non-limiting example, hydroxyzine HCl, hydroxyzine pamoate, cyclizine HCl, cyclizine lactate, meclizine HCl, and cetirizine HCl. The compositions of the present invention for the treatment of nicotine withdrawal symptoms comprise formulations about 0.25 cc to about 6 cc in volume, wherein hydroxyzine is present in an amount ranging from about 0.1 mg to about 100 mg.

In some embodiments of the invention, the anticholinergic medication optionally comprises, one or more of another anticholinergic agent in a therapeutically effective amount. As used herein, the term “anticholinergic agent” means any compound or composition having anticholinergic properties. Suitable anticholinergic agents are those compounds that act as antagonists at the muscarinic receptor, in particular those compounds which are antagonists of the M₁, M₂, or M₃ receptors, or of combinations thereof. Exemplary compounds include the alkaloids of the belladonna plants as illustrated by the likes of atropine, scopolamine, homatropine, hyoscyamine; these compounds are normally administered as a salt, being tertiary amines. These drugs, particularly the salt forms, are readily available from a number of commercial sources or can be made or prepared from literature data via, to wit:

Atropine, anhydrous form (CAS-51-55-8 or CAS-51-48-1), atropine sulfate (CAS-5908-99-6); atropine oxide (CAS-4438-22-6) or its HCl salt (CAS-4574-60-1) and methylatropine nitrate (CAS-52-88-0).

Homatropine (CAS-87-00-3), hydrobromide salt (CAS-51-56-9), methylbromide salt (CAS-80-49-9).

Hyoscyamine (d, 1) (CAS-101-31-5), hydrobromide salt (CAS-306-03-6) and sulfate salt (CAS-6835-16-1).

Scopolamine (CAS-51-34-3), hydrobromide salt (CAS-6533-68-2), methylbromide salt (CAS-155-41-9).

Other anticholinergic agents useful in the present invention are: glycopyrollate, a synthetic muscarinic receptor antagonist, ipratropium (e.g. as the bromide), sold under the name ATROVENT, oxitropium (e.g. as the bromide), tiotropium (e.g. as the bromide) (CAS-139404-48-1), methantheline (CAS-53-46-3), propantheline bromide (CAS-50-34-9), anisotropine methyl bromide or VALPIN 50 (CAS-80-50-2), clidinium bromide (QUARZAN, CAS-3485-62-9), copyrrolate (ROBINUL), isopropamide iodide (CAS-71-81-8), mepenzolate bromide (U.S. Pat. No. 2,918,408), tridihexethyl chloride (PATHILONE, CAS-4310-35-4), and hexocyclium methylsulfate (Tral, CAS-115-63-9). See also cyclopentolate hydrochloride (CAS-5870-29-1), tropicamide (CAS-1508-75-4), trihexyphenidyl hydrochloride (CAS-144-11-6), pirenzepine (CAS-29868-97-1), telenzepine (CAS-80880-90-9), AF-DX 116, oxybutynin and methoctramine.

Preferably, the anticholinergic medication optionally provides for one or more of a therapeutically effective amount of an anticholinergic agent selected from the group consisting of atropine, homatropine, hyoscyamine and glycopyrrolate. The compositions of the present invention for the treatment of nicotine withdrawal symptoms comprise formulations about 0.25 cc to about 6 cc in volume, wherein atropine is present in an amount ranging from about 0.01 mg to about 0.6 mg, homatropine is present in an amount ranging from about 0.01 to about 0.8 mg, hyoscyamine is present in an amount from ranging from about 0.01 mg to about 0.8 mg, and glycopyrrolate is present in an amount ranging from about 0.01 mg to about 0.4 mg.

Most preferably, the anticholinergic medication optionally comprises hyoscyamine. Hyoscyamine is an anticholinergic agent which is solely responsible for the antimuscarinic action of atropine. Since the predominance of nicotinic receptors are located centrally at the mid-brain level, without wishing to be bound by theory, it is postulated that hyoscyamine is more effective than atropine in smoking cessation. The compositions of the present invention comprise formulations about 0.25 cc to about 6 cc in volume, wherein hyoscyamine is present in an amount ranging from about 0.01 mg to about 0.8 mg.

In addition, pharmaceutically acceptable carriers may be used in the compositions of the present invention. The term “pharmaceutically acceptable carrier” refers to a carrier that may be administered to a patient, together with scopolamine and hydroxyzine, and optionally, one or more of atropine, chlorpromazine, homatropine, hyoscyamine and/or glycopyrrolate, and which does not destroy the pharmacological activity thereof and is nontoxic when administered in doses sufficient to deliver a therapeutic amount of the compositions of the present invention.

Non-limiting exemplary examples of pharmaceutically acceptable carriers include, ion exchangers, alumina, aluminum stearate, lecithin, self-emulsifying drug delivery systems (SEDDS) such as d alpha-tocopherol polyethyleneglycol 1000 succinate, or other similar polymeric delivery matrices or systems, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-.beta.-cyclodextrins, or other solublized derivatives may also be advantageously used to enhance delivery of therapeutically-effective scopolamine and hydroxyzine, and optionally, one or more of atropine, chlorpromazine, homatropine, hyoscyamine and/or glycopyrrolate.

The compositions of the present invention may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like.

The compositions of the present invention may also include anesthetics such as lidocaine, marcaine or procaine to reduce any burning associated with administration of the compositions by injection.

As used herein, “therapeutically effective amount” or “therapeutically effective dose” means a nontoxic but sufficient amount of an active agent to provide the desired therapeutic effect (i.e., minimizing nicotine withdrawal symptoms). It will be understood, however, that the total usage (daily or otherwise) of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved.

The pharmaceutical compositions of the present invention may be administered parenterally, orally, intracisternally, intravaginally, intraperitoneally, topically (as by powders, ointments, drops or transdermal patch), rectally, or bucally. The term “parenteral” as used herein refers to modes of administration which include intravenous, intramuscular, intraperitoneal, intrasternal, subcutaneous and intraarticular injection and infusion. In a preferred embodiment, the pharmaceutical compositions of the present invention comprising an anticholinergic medication is administered intramuscularly.

Pharmaceutical compositions for parenteral injection comprise pharmaceutically acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

Injectable depot forms are made by forming microencapsule matrices of the drug in biodegradable polymers such as polylactide-polyglycolide, poly(orthoesters), poly(anhydrides), and (poly)glycols, such as PEG. Depending upon the ratio of drug to polymer and the nature of the particular polymer employed, the rate of drug release can be controlled. Depot injectable formulations are also prepared by entrapping the drug in liposomes or microemulsions which are compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Prolonged absorption of the injectable formulations may be brought about by the inclusion of agents which delay absorption, such as aluminum monostearate and gelatin.

The pharmaceutical compositions of the present invention may be in a form suitable for transdermal administration. By “transdermal” drug delivery is meant administration of a drug to the skin surface of an individual so that the drug passes through the skin tissue and into the individual's blood stream, thereby producing a systemic effect. The term “transdermal” is intended to include “transmucosal” drug administration, i.e., administration of a drug to the mucosal (e.g., sublingual, buccal, vaginal, rectal) surface of an individual so that the drug passes through the mucosal tissue and into the individual's blood stream.

The pharmaceutical compositions of the present invention may be in a form suitable for oral use, for example, as tablets, lozenges, aqueous or oily suspensions, dispersible powders or granules, emulsions, hard or soft capsules, syrups or elixirs. Compositions intended for oral use may be prepared according to any method known to the art for the manufacturer of pharmaceutical compositions and such compositions may contain one or more agents such as, for example, sweetening agents, flavoring agents, coloring agents and the like, in order to provide a pharmaceutically elegant and palatable preparation.

Tablets contain the active ingredients in admixture with nontoxic pharmaceutically acceptable excipients which are suitable for manufacture of tablets. These excipients may be, inert diluents, for example, calcium carbonate, sodium carbonate, lactose, calcium phosphate or sodium phosphate; granulating and disintegrating agents, for example, alginic acid, croscarmellose sodium, maize starch or; binding agents, for example, acacia, gelatine or starch, and lubricating agents, for example, magnesium stearate or stearic acid. The tablets may be uncoated or they may be coated by known techniques to delay disintegration and absorption in the gastro-intestinal tract and thereby provide an even longer sustained action over a period of time. The tablets may be chewable or non-chewable and designed to desired weight, potency and hardness through well known skills in the pharmaceutical arts.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with a suitable oil medium, for example, arachis oil, liquid paraffin or olive oil.

Formulations for oral use may also be presented as lozenges wherein the active ingredients are mixed into a hard candy composition. Suitable hard candy compositions can be made from varying, but highly concentrated, sucrose solutions including corn syrup as a second essential ingredient. Other known hard candy compositions may utilize any suitable good testing, sweet excipient other than sucrose.

Aqueous suspensions contain the active ingredients in admixture with excipients suitable for the manufacture of aqueous suspensions. Such excipients or combinations thereof may be suitable suspending agents, for example, alginates, carboxymethylcellulose, carboxypolymethylene, carrageenan, colloidal silcon dioxide, corn starch, flowable starch, gelatin, guar gum, gum acacia, gum tragacanth, hydroxypropylcellulose, hydroxypropylmethylcellulose, maltodextrin, methylcellulose, microcrystalline cellulose, pectin, polyethylene glycol 800, polyvinyl alcohol, polyvinylpyrrolidone, sodium alginate, sodium carboxymethyl cellulose or xanthum gum; dispersing or wetting agents may be any suitable naturally occurring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol, for example, polyoxyethylene sorbitol monnoleate, or condensation product of ethylene oxide with partial esters derived from fatty acids and hexitol and anhydrides, for example, polyoxyethelyne sobirtan monooleate, or water. The aqueous suspensions may also contain one or more suitable preservatives, for example, ethyl, or n-propyl, p-hydroxy benzoate, one or more suitable coloring agents, one or more suitable flavoring agents such as, cinnamon, chocolate, fruit flavors (i.e., cherry, grape, orange, strawberry, etc.), menthol, mints, vanilla and combination of two or more thereof, one or more suitable sweetening agents, such as calcium cyclamate, dextrose, fructose, galactose, glucose, glycerin, maltose, mannitol, mannose, ribose, partially hydrolyzed starch solids, partially hydrolyzed corn syrup solids, sodium cyclamate, sorbital, inulin, sucralose, sucrose, xylitol, or xylose, and one or more suitable coloring agents.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents may be exemplified by those already mentioned above. Additional suitable excipients, for example, sweetening, flavoring and coloring agents, may also be present.

Syrups and elixirs may be formulated with suitable sweetening agents, for example, one or more of glycerol, sorbitol, inulin, sucrose or xylose. Such formulations may also contain suitable demulcents, preservatives such as citric acid and flavoring and coloring agents.

The individual compounds (e.g., scopolamine and hydroxyzine) of the pharmaceutical compositions may be administered either substantially together or simultaneously in separate or combined pharmaceutical formulations. By “substantially together”, the active ingredients of the composition of the invention are administered to a patient in separate dosage forms, such that, the active ingredients are administered either simultaneously or within a period of time such that the patient receives benefit of the aggregate effects of the separate dosage forms. For example, the active ingredients may be taken together or within a few seconds to at least about 30 minutes of one another. Accordingly, methods of treatment of the present invention, therefore, include administration of the individual compounds of such combinations either substantially together or simultaneously in separate or combined pharmaceutical formulations. When administering or taking the active ingredients substantially together, but separately in same or different dosage forms, the order in which they are administered or ingested is not critical.

Thus, according to a further aspect, the invention provides a method of treating nicotine withdrawal symptoms associated with smoking cessation, comprising administering to a patient in need thereof, a therapeutically effective amount of an anticholinergic medication comprising scopolamine and hydroxyzine, and optionally, a therapeutically effective dose of one or more of an anticholinergic agent, preferably selected from the group consisting of atropine, homatropine, hyoscyamine and glycopyrrolate. In one embodiment, the method further comprises chlorpromazine. In a preferred embodiment, a patient is treated by injecting the patient with the anticholinergic medication of the present invention. Preferably, the anticholinergic medication is administered by intramuscular injection to the buttocks or triceps muscles. The anticholinergic medication may be given in a single dose or in two half doses during a single office visit. Administration of a single dose eliminates the need for titration. Typically, a therapeutic dose ranges from about 1.8 cc to about 3.0 cc, and preferably is about 2.0 cc.

In another embodiment of the present invention, in place of an injection, the anticholinergic medication is administered, simultaneously or substantially together, transdermally via, for example, a scopolamine patch, and/or orally via, for example, belladonna tablets and hydroxyzine tablets according to accepted pharmacological principals.

While the anticholinergic injection is designed to obtain immediate and high levels of anticholinergic activity, a maintenance regime is designed to maintain lower levels of anticholinergic activity during the remainder of the withdrawal period. In another embodiment, the method of the present invention further comprises a step of continuing the effects of the anticholinergic medication administered via injection. Oral and transdermal anticholinergic agents are prescribed and subsequently ingested by the patient to provide continued relief from excessive acetylcholine associated with nicotine withdrawal until the physiological effects of nicotine withdrawal are sufficiently minimized. Such non-limiting exemplary oral anticholinergic agents include scopolamine tablets, belladonna tablets, donnatal (mixture of Phenobarbital, hyoscyamine, atropine and scopolamine) tablets, hydroxyzine tablets, and oxybutynin tablets, or propantheline tablets. Such non-limiting exemplary transdermal anticholinergic agents include scopolamine patches. Preferably, the patient begins taking the prescribed anticholinergic agent the day following receipt of the injected anticholinergic medication and continues for about two weeks, or until, nicotine withdrawal symptoms are sufficiently minimized. While this method addresses the physiological aspects of nicotine withdrawal; however, there are also psychological associations linked to smoking cigarettes which must be overcome for successful smoking cessation.

The invention thus provides in further embodiment, a step for alleviating anxiety associated with nicotine withdrawal. Anxiolytic agents are prescribed using accepted pharmacological principals and subsequently ingested as prescribed by a patient. As used herein “anxiolytic agent” means any compound or composition having anxiolytic properties. Non-limiting examples of an anxiolytic include, abecarnil, alpidem, benzodiazepines, buspirone, chlormezanone, chlorpromazine, clonidine, flupirilene, hydroxyzine, trandospirone and meprobamate. Preferably, the patient begins taking the anxiolytic agent following receipt of the anticholinergic medication. Alternatively, the anxiolytic agent can be administered simultaneously or substantially together with the anticholinergic medication, for example, combined into and administered as a component of the anticholinergic medication administered by injection.

The invention also provides in further embodiment, a step for alleviating depression which can also be associated with nicotine withdrawal. Antidepressants are prescribed using accepted pharmacological principals and subsequently ingested as prescribed by a patient. Preferably, antidepressants such as serotonin reuptake inhibitors, bupropion and buspirone are prescribed prior to or in conjunction with a patients receipt of the anticholinergic medication.

In a further embodiment, the invention also provides, several methods of disassociating the habits of smoking by implementing a behavioral modification program and/or hypnosis. These associations may be addressed prior to administration of the anticholinergic medication in order to enhance the success of a smoking cessation program. Unfortunately, many patients resume smoking well after the physiological addiction has resolved. Usually this is due to poor coping habits and lack of support. Therefore, smoking cessation success rates may also be enhanced by routine support and counseling after the physiologic withdrawal has resolved. Non-limiting examples of the formats in which smoking cessation support and counseling is offered include, telephone conference calls, internet discussion boards, internet chat rooms, group meetings and individualized counseling sessions. Counseling and support may also be provided to a patient through various media, such as video tapes, CDs, DVDs, audiotape, having recorded thereon various educational and support materials on and for the cessation of smoking.

In order to further illustrate the present invention and the advantages thereof, the following specific non-limiting Examples are given, it being understood that these Examples are intended only to be illustrations without serving as a limitation on the scope of the present invention.

### EXAMPLE 1

Prior to the office visit, a patient completes a medical questionnaire and detailed smoking history questionnaire. If there is no contraindication to anticholinergic medications, an appointment is scheduled. Any patient with contraindications to the anticholinergic medication will be excluded from receiving the shot and alternative therapies such as hypnosis, will be offered. Prior to the office appointment, the patient is asked to review a smoking cessation video or other media and to read associated smoking cessation literature. In addition, there is a series of “homework” steps assigned to the patient designed to disassociate the routine habits of smoking from the cigarette smoking process. Non-limiting examples of such homework steps include, smoking only in one area of the house, and not taking part in any activity while smoking.

During the office visit, a physician performs a history and physical examination of the patient. In addition, counseling may be provided, as well as, any ancillary tests such as spirometry or electrocardiogram, on an as needed basis, to rule out any contraindications.

Patients determined to be suited for the anticholinergic medication receive an intramuscular injection of about ½ of the amount of about a 0.25 cc to about 6 cc solution comprising from about 0.01 mg to about 0.8 mg of hyoscyamine, from about 0.01 mg to about 0.8 mg of scopolamine, and from about 0.1 mg to about 100 mg of hydroxyzine to one of the buttocks. Patients are then allowed to sit in a darkened room and re examined after 5-15 min. Patients without excessive xerostomia and with normal pupillary constriction may then receive the remaining solution injected to the other buttocks. The physician may choose to alter the components of the remaining solution for the second injection depending upon findings on the physical exam. The patient is then observed in the office until stable for discharge. The patient is instructed not to drive or drink alcohol within 8 hours of the injection. The next morning, the patient is started on anticholinergic agents such as scopolamine patches, belladonna tablets, donnatal, hydroxyzine tablets, oxybutynin, or propantheline tablets, to continue the anticholinergic block for up to 14 days.

Though this acetylcholine anticholinergic block may be successful without an further follow up, many patients need to conquer the psychological addiction of smoking. To address this psychological addiction numerous methods of support such as telephone counseling, internet discussion boards, internet chat rooms, group telephone conference calls and group meetings may be implemented. As needed, anxiolytics or antidepressants may also be prescribed.

### EXAMPLE 2

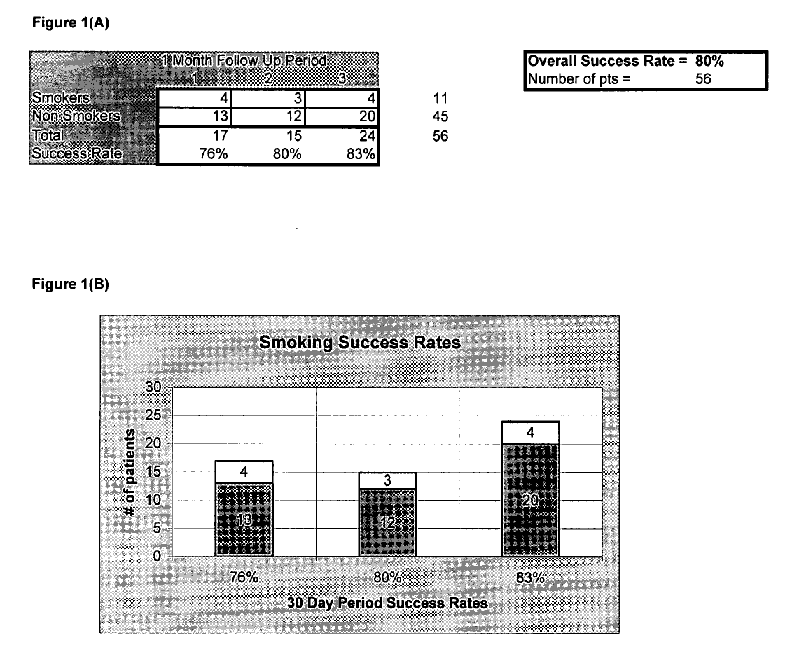

Three groups of adult smokers received via intramuscular injection to the buttocks and/or triceps a single dose of about 2 cc of an anticholinergic medication comprising about 0.4 mg of scopolamine and 25 mg of hydroxyzine. The patients were observed in the office for about 15 minutes prior to discharging the patient home. FIG. 1(A) and (B) graphically illustrate the percentage of patients who successfully stopped smoking and continued to be nonsmokers 30 days after having received the anticholinergic medication. On average, 80% of those treated remain nonsmokers.

Although illustrative embodiments of the present invention have been described in detail, it is to be understood that the present invention is not limited to those precise embodiments, and that various changes and modifications can be effected therein by one skilled in the art without departing from the scope and spirit of the inventions as defined by the appended claims.

1. An anticholinergic medication for the treatment of nicotine withdrawal associated with smoking cessation in a patient in need thereof, comprising a pharmaceutically effective amount of scopolamine and hydroxyzine.

2. The anticholinergic medication according to claim 1 wherein scopolamine is present in an amount from about 0.01 mg to about 0.8 mg.

3. The anticholinergic medication according to claim 1 wherein hydroxyzine is present in an amount from about 0.01 mg to about 100 mg.

4. The anticholinergic medication according to claim 1 further comprising a pharmaceutically effective amount of an anticholinergic agent selected from the group consisting of atropine, homatropine, hyoscyamine, and glycopyrrolate.

5. The anticholinergic medication according to claim 4 wherein the anticholinergic agent is atropine.

6. The anticholinergic medication according to claim 5 wherein atropine is present in an amount from about 0.01 mg to about 0.8 mg.

7. The anticholinergic medication according to claim 4 wherein the anticholinergic agent is homatropine.

8. The anticholinergic medication according to claim 7 wherein homatropine is present in an amount from about 0.01 mg to about 0.8 mg.

9. The anticholinergic medication according to claim 4 wherein the anticholinergic agent is hyoscyamine.

10. The anticholinergic medication according to claim 9 wherein hyoscyamine is present in an amount from about 0.01 mg to about 0.8 mg.

11. The anticholinergic medication according to claim 4 wherein the anticholinergic agent is glycopyrrolate.

12. The anticholinergic medication according to claim 11 wherein glycopyrrolate is present in an amount from about 0.01 mg to about 0.4 mg.

13. The anticholinergic medication according to claim 1 further comprising a pharmaceutically acceptable carrier.

14. The anticholinergic medication according to claim 1 administered by intramuscular injection.

15. An anticholinergic medication for the treatment of nicotine withdrawal associated with smoking cessation in a patient in need thereof, comprising a pharmaceutical solution administered by injection comprising scopolamine present in an amount from about 0.01 mg to 0.8 mg, hydroxyzine present in an amount from about 0.01 mg to about 100 mg, and a pharmaceutically acceptable carrier.

16. The anticholinergic medication according to claim 15 further comprising one or more of an anticholinergic agent selected from the group consisting of atropine present in an amount from about 0.01 mg to about 0.8 mg, homatropine present in an amount from about 0.01 mg to about 0.8 mg, hyoscyamine present in an amount from about 0.01 mg to about 0.8 mg, and glycopyrrolate present in an amount from about 0.01 mg to about 0.4 mg.

17. A method of using a pharmaceutical composition comprising scopolamine and hydroxyzine, and optionally, one or more of atropine, chlorpromazine, homatropine, hyoscyamine and glycopyrrolate for the treatment for nicotine withdrawal symptoms associated with smoking cessation comprising administering to a patient in need thereof a therapeutically effective amount of the pharmaceutical composition.

18. A method for reducing nicotine withdrawal symptoms associated with smoking cessation in a patient in need thereof comprising, injecting the patient with a pharmaceutical solution of about 0.25 cc to about 6.0 cc comprising scopolamine present in an amount from about 0.01 mg to about 0.8 mg, hydroxyzine present in an amount from about 0.01 mg to about 100 mg, and a pharmaceutically acceptable carrier.

19. The method according to claim 18 wherein the pharmaceutical solution further comprises an anticholinergic agent selected from the group consisting of atropine present in an amount from about 0.01 mg to about 0.8 mg, homatropine in an amount from about 0.01 mg to about 0.8 mg, hyoscyamine present in an amount from about 0.01 mg to about 0.8 mg, and glycopyrrolate present in an amount from about 0.01 mg to about 0.4 mg.

20. The method according to claim 18 further comprising the concomitant prescription to the patient of a therapeutically effective dose of one or more of an anticholinergic agent selected from the group consisting of atropine, belladonna, homatropine, hyoscyamine, hydroxyzine, glycopyrrolate, oxybutynin, propantheline and scopolamine using accepted pharmacological principals.

21. The method according to claim 18 further comprising the concomitant prescription to the patient of a therapeutic dose of benzodiazepine, chlorpromazine, clonidine or hydroxyzine.

22. The method according to claim 18 further comprising prescribing to the patient an antidepressant.

23. The method according to claim 18 further comprising implementing a behavioral modification therapy.

24. The method according to claim 23 wherein the behavioral modification therapy is selected from one or more of the group of hypnosis, individual counseling, group counseling, and streaming educational media.

25. The method according to claim 18 wherein the pharmaceutical solution is administered by a single injection to the buttocks or triceps.

26. The method according to claim 18 wherein one half of the amount of the pharmaceutical solution is administered by intramuscular injection to the patient's buttocks and the second half of the pharmaceutical solution is injected to the patient's other buttocks.
